(19) United States
(12) Patent Application Publication
(10) Pub. No.: US 2025/0320536 A1
(43) Pub. Date: Oct. 16, 2025

(54) CARBON INTENSITY OF FIRST-GENERATION BIOETHANOL

(71) Applicant: SixRing Inc., Calgary (CA)

(72) Inventors: Alejandra Enriquez, Calgary (CA); Scott Anthony Treadwell, Calgary (CA); Markus Weissenberger, Calgary (CA); Julie Greer, Calgary (CA)

(21) Appl. No.: 19/176,397

(22) Filed: Apr. 11, 2025

(30) Foreign Application Priority Data
Apr. 12, 2024 (CA) 3235094

(51) Int. Cl. C12P 19/04; C08H 8/00; C12N 9/42

(52) U.S. Cl. CPC C12P 19/04; C08H 8/00; C12N 9/2437; C12N 9/2445; C12Y 302/01004; C12Y 302/01021; C12Y 302/01091

(57) ABSTRACT

A process to manufacture a value added product by blending a stream of cellulose-based hydrolysate with a non-cellulose based hydrolysate, wherein said process comprising the steps of:
providing a high purity cellulose comprising of less than 1.5% lignin;
exposing said high purity cellulose to a saccharification process to produce a cellulosic hydrolysate comprising sugars obtained from the hydrolysis of cellulose and hemicellulose;
exposing said cellulosic hydrolysate to another sugar hydrolysate obtained from a saccharification of a non-cellulose based sugar source material, thus obtaining a combined hydrolysate stream;
processing said combined hydrolysate stream to produce SAID value-added product; and
optionally, purifying and/or separating said at least one value-added product from the rest of the fermentation stream to yield a purified value-added product.

### FIELD OF THE INVENTION

The present invention is directed to a process to substantially decrease the carbon intensity of first-generation ethanol production through the use of a specific cellulose that is derived from a highly efficient delignification process.

### BACKGROUND OF THE INVENTION

Biofuels are increasingly becoming a necessity in order to reduce the human consumption of fossil fuels in aspects of everyday life, transport and home heating being the largest two industries of focus. As an alternative energy source to oil and coal, the main feedstock for bioethanol production is starch which can yield its sugar much more readily than cellulose. This is due to the difference in structure as starch links glucose molecules together through alpha-1,4 linkages and cellulose links glucose with beta-1,4 linkages. The beta-1,4 linkages allow for crystallization of the cellulose, leading to a more rigid structure which is more difficult to break down.

The limitation that comes from solely using the sugars from starches for the production of biofuels such as bioethanol prevents the utilization of the larger portion of biomass, which comes in the form of lignocellulosic biomass (contains lignin, cellulose and hemicellulose) present in almost every plant on earth. A delignification reaction allows the recovery of the carbohydrate-based portion (cellulose and hemicellulose) from those lignocellulosic plants. Once the cellulose is separated from the other two biomass constituents i.e., lignin, and hemicellulose, further degradation of the cellulose generates oligosaccharides (i.e., glucose) which can be further processed to bioethanol.

Seen as a sustainable alternative to gasoline and with the goal of alleviating many countries' dependence on foreign oil, the bioethanol industry is still hampered by its dependence on corn or sugar cane as its main sources of biomass, as they are both rich in starch. It is estimated that about 45% of all corn production in the U.S. is directed to the ethanol fuel production. This is a situation which has disastrous consequences when the prices of gasoline go so low as to make corn-based biofuel unsustainable on a price viewpoint.

Across the world, many other large bioethanol-producing countries, including China and Brazil, have shown some struggles in ethanol production from biomass as many companies are carrying large debts from the implementation of such processes and large plants have been forced to shut down or decrease production.

For society to move away from burning fossil fuels, it is not enough to make renewable biofuels available for use. These alternatives need to also provide significant economic and environmental advantages including but not limited to more efficient combustion/use, more cost-effective production, reduction in greenhouse gas emissions, etc. Lifecycle assessments are tools designated to identify and measure the environmental impact of new technologies. They focus on determine savings in energy and emissions throughout the entire production cycle of the alternative fuel. Estimates for the carbon intensity (CI) of corn ethanol over the past three decades range from 52 to 105 grams of carbon dioxide equivalent emission per megajoule of energy (gCO₂e/MJ). The approved fuel pathways database for California's LCFS program reports GHG emissions intensities (CI scores) for corn-only dry mill ethanol facilities ranging between 53 and 86 gCO₂e/MJ, while the mean certified CI is 70.2 gCO₂e/MJ.

First-generation (1G) biofuels are those obtained from the processing of edible materials (i.e., corn, sugar beets, molasses, etc.), while second-generation biofuels are defined as fuels produced from feedstock that is not in competition with food production (i.e., lignocellulosic biomass, municipal solid wastes, etc.). Some of the main drawbacks of the use of 1G biofuels include the impact on food prices and availability worldwide as well as environmental impacts related to the use of arable land for biofuel production.

To pivot from starches/sugars (1G) to cellulose (2G—“second generation”) for the production of bioethanol is preferable as it will provide near-unlimited amounts of feedstock from waste biomass and reduce the competition with food to generate bioethanol. However, the costs to do so are currently prohibitive. Cellulosic ethanol as it is called relies on the non-food part of a plant to be used to generate ethanol. This would allow the replacement of the currently widespread approach of making bioethanol by using corn or sugarcane. The diversity and abundance of these types of cellulose-rich plants would keep production of food resources mostly intact and capitalize on the waste generated from these food resources (such as cornstalk) to generate ethanol. Other cellulose sources such as grasses, algae and even trees fall under the category of cellulose-rich biomass which can be used in generating ethanol if a commercially viable process is developed.

The reason why starches are preferred to cellulose-rich sources to generate ethanol is that extraction of glucose from cellulose is substantially more difficult and resource intensive. To better understand the difference which raises this difficulty it is worthwhile pointing the similarities and differences between starch and cellulose.

Cellulose and starch are polymers which have the same repeat units of glucose. However, the differences between starch and cellulose can be seen in the way the repeating glucose monomers are connected to one another. In starch, the glucose monomers are oriented in the same direction. In cellulose, each successive glucose monomer is rotated 180 degrees in respect of the previous glucose monomer. This, in turn, ensures that the bonds between each monomeric glucose differs between starch and cellulose. In starch, the bonds (otherwise known as links) are referred to as α-1,4 linkages, in cellulose these bonds are referred to as β-1,4 linkages.

The difference between these bonds impacts the characteristics of starch and cellulose. Starch can dissolve in warm water while cellulose does not. Starch can be digested by humans, cellulose cannot. Starch is weaker than cellulose partly due to the fact that its structure is less crystalline than cellulose. Starch is, at its core, a method for plants to store energy, therefore extracting sugars from starch is much easier than to do so from cellulose as the latter's core function is to provide structural support.

As the main component of lignocellulosic biomass, cellulose is a biopolymer consisting of many glucose units connected through β-1,4-glycosidic bonds. D-glucose is the building block of many polysaccharides, including cellulose. Glucose has two isomers: α-glucose (present in starches as branched polymers) and β-glucose (present in cellulose as repeating units of β-glucose subunits connected via a β-1,4-glycosidic bond with one β-glucose monomer rotated by 180 degrees relative to its neighbour). A cellulose molecule can comprise between hundreds to thousands of glucose units. Since the cellulose molecules are linear, due in part to intermolecular hydrogen bonding, neighboring cellulose molecules can be very closely packed and, in turn, provide the structural strength needed to support plants.

The ability to integrate first generation (1G) and second generation (2G) ethanol facilities is viewed as advantageous as it increases the yield of ethanol from the same area (no need for land expansion) and it lowers climate change impacts. According to a preferred embodiment of the present invention, the process comprises the presence of a first generation (1G) facility and second generation (2G) ethanol facility on the same site. According to another preferred embodiment of the present invention, the sugars obtained from the delignification of the lignocellulosic biomass can be shipped to a different site than the one where delignification occurred.

In the paper entitled “A new insight into integrated first and second-generation bioethanol production from sugarcane” (Industrial Crops and Products, 2022, Volume 188, Part A, 115675), the authors study the economic feasibility of seven process configurations for integrated molasses (1 G) and lignocelluloses (2 G) bioethanol production, in combined first-and second-generation (1G2G) facilities annexed to a typical South African sugar mill. Simulations for various first generation (1 G-only), second-generation (2 G-only) and integrated 1G2G biorefinery scenarios were developed and rigorous techno-economic and sensitivity analyses were conducted. The results determined that production of 1 G-only ethanol from C-molasses obtained a better minimum ethanol selling price (MESP) of 0.68/$/1, compared to A-molasses (1.05 $/L) due to the significantly higher price of A-molasses (314 $/t) relative to C-molasses (192 $/t). The integration of 1 G and 2 G sugars for 1G2G ethanol production showed significant economic benefits (up to 50% improvement), compared to 2 G-only ethanol production, thereby lowering the cost of lignocelluloses conversion. The 1G2G scenario that produced the most favourable MESP of 1.23 $/litre involved the supplementation of the whole slurry of pre-treated lignocelluloses (2 G) with A-molasses (1 G) for co-fermentation of sugars by Separate Hydrolysis and Co-fermentation (SHcF). Despite technical differences between scenarios 1G2G1, 1G2G3 and 1G2G7, no significant differences could be observed in terms of MESPs (1.25, 1.23 and 1.26 $/L, respectively), which were the lowest value among all integrated scenarios. The main drivers of the outstanding economic performance were (1) the ability of the selected fermenting microorganism to function at sufficiently high substrate concentrations without inhibition or glucose suppression, (2) economies-of-scale benefits, (3) the high yield of sugar utilization and (4) the choice of optimum process condition for co-fermentation of C5 and C6 sugars.

In the paper titled “Comparative life cycle assessment of first-and second-generation ethanol from sugarcane in Brazil” (Int. J. Life Cycle Assess. 24, 266-280 (2019)), the authors investigated and quantified different technological options of ethanol production looking at potential environmental impacts of the use of bagasse and trash from sugarcane fields in ethanol production. The first-generation ethanol from sugarcane is compared to stand-alone second-generation ethanol as well as an integrated first-and second-generation ethanol production.

US patent application 2015/0064762A1 refers to a system and a process for the production of ethanol and related products from lignocellulosic biomasses (second generation 2G-ethanol), particularly from Sugarcane bagasse and straw, however not limited thereto, integrated with conventional processes for the production of ethanol (first generation—1G-ethanol) such as, for example, from Sugarcane juice and/or molasses (a process that is typically Brazilian, either in Sugar and ethanol plants or in autonomous distilleries), corn, grain, wheat, Sugary Sorghum, white beetroot, among others, comprising the recovery/reuse of streams and effluents. More specifically, the present invention refers to an integrated process for the production of ethanol and related products where the said process provides an increased efficiency particularly in the use of the raw material, steam, electric power and treated water.

US patent application 2023/0076406 is directed to an optimized process for the production of ethanol from energy cane, by the integration of first-generation (1G) and second-generation (2G) technologies, which presents the advantages of reducing energy and water consumption. More specifically, the secondary juice from the second set of three rolls of mills of the conventional process (1G) is used for the dilution, in the enzymatic hydrolysis step, in the cellulosic ethanol production process (2G).

US patent application number 2021/0403958 A1 discloses a method whereby ethanol is produced by the simultaneous production of both First and Second generation (1G, 2G) fuel grade ethanol in the same production plant. A First-Generation feedstock such as corn is continuously fed to the first-generation section and a lignocellulosic feedstock such as corn stover from the 1G corn is supplied to the second-generation area. Thus, there is a common fermentation area for both the C5 and C6 sugar fermentation. The invention can economically be best implemented in places where there are incentives offered for the use of various feedstocks. Specifically, the invention allows the D3 RIN (Renewable Identification Number) to be maximized in an existing first-generation ethanol plant with the installation of the front end of the 2G equipment.

In light of the above, there exists an unmet need to develop a process for biofuel or bioethanol generation from a combination of first-generation and second-generation feedstocks that can significantly lower the carbon intensity of the first-generation process and maximize the yield obtained from the non-food part of the lignocellulosic biomass. Additionally, there is a need for a process that utilizes a high purity cellulose as the starting material for the 2G saccharification process as it provides clear advantages, namely the increase in efficiency due to the lack of inhibitors typically obtained from the pretreatment of biomass (i.e., inhibitors from lignin and hemicellulose).

The inventors have surprisingly and unexpectedly found that the characteristics of the cellulose obtained from a specific type of delignification approach have a substantial impact on the downstream hydrolysis of said cellulose to glucose and that the process is extremely sustainable and efficient, leading to considerably lower greenhouse gas emissions.

### SUMMARY OF THE INVENTION

According to an aspect of the present invention, there is provided a process to obtain ethanol from sugar fermentation by blending a sugar hydrolysate stream mainly comprised of glucose obtained from the hydrolysis of a high purity cellulose with a sugar hydrolysate stream obtained from the hydrolysis of a mainly starch-based stream.

According to an aspect of the present invention, there is provided a process to reduce the overall energy input in the preparation of ethanol by the fermentation of various sugar hydrolysates from different sources, wherein said process comprises a step of blending a sugar hydrolysates stream mainly comprised of glucose obtained from the hydrolysis of a high purity cellulose with a sugar hydrolysate stream obtained from the hydrolysis of a mainly starch-based substrate.

According to an embodiment of the present invention, there is provided a process to obtain a low-carbon-intensive combined hydrolysate stream, said process comprising the steps of:
providing a high purity cellulose comprising of less than 1.5% lignin;
exposing said high purity cellulose to a saccharification process to produce a cellulosic hydrolysate comprising sugars obtained from the hydrolysis of cellulose and hemicellulose;
exposing said cellulosic hydrolysate to another sugar hydrolysate obtained from a saccharification of a non-cellulose based sugar source material, thus obtaining a combined hydrolysate stream;
processing said combined hydrolysate stream to produce at least one value-added products; and
optionally, purifying and/or separating said at least one value-added product from the rest of the fermentation stream to yield a purified value-added product.

According to an aspect of the present invention, there is provided a process to manufacture a value added product by blending a stream of cellulose-based hydrolysate with a non-cellulose based hydrolysate, wherein said process comprising the steps of:
providing a lignocellulosic biomass;
exposing said lignocellulosic biomass to a delignification process whereby a high purity cellulose is obtained; said high purity cellulose comprising of less than 1.5% lignin;
exposing said high purity cellulose to a saccharification process to produce a cellulosic hydrolysate comprising sugars obtained from the hydrolysis of cellulose and hemicellulose;
exposing said cellulosic hydrolysate to another sugar hydrolysate obtained from a saccharification of a non-cellulose based sugar source material, thus obtaining a combined hydrolysate stream;
processing said combined hydrolysate stream to produce at least one value-added product; and
optionally, purifying and/or separating said at least one value-added product from the rest of the fermentation stream to yield a purified value-added product.

According to an aspect of the present invention, there is provided a process to manufacture a value added product by blending a stream of cellulose-based hydrolysate with a non-cellulose based hydrolysate, wherein said process comprising the steps of:
providing a lignocellulosic biomass;
exposing said lignocellulosic biomass to a modified Caro's acid composition for a period of time necessary to remove more than 98.5% of the lignin present in said lignocellulosic biomass and thus obtaining a high purity cellulose and a liquid stream;
exposing said high purity cellulose to a saccharification process to produce a cellulosic hydrolysate comprising sugars resulting from the hydrolysis of cellulose and hemicellulose;
exposing said cellulosic hydrolysate to another sugar hydrolysate obtained from the saccharification of a non-cellulose-based sugar source, thus obtaining a combined hydrolysate stream;
processing said combined hydrolysate stream to produce said value-added product; and
optionally, purifying said at least one value-added product to yield a purified value-added product.

In some embodiments of the present invention, the process of exposing said lignocellulosic biomass to a modified Caro's acid composition can be carried out for a varying duration of time depending on the particle size of the biomass and the type of biomass being fed into the process. In some cases, the process can last from 2 to 20 hours depending on that characteristic. The process is preferably run at temperatures below 50° C., more preferably at temperatures below 40° C.

According to a preferred embodiment of the present invention, the cellulosic sugar hydrolysate is combined with the another sugar hydrolysate obtained from the saccharification of a non-cellulose based sugar source material in a weight ratio ranging from 99:1 to 1:99. Preferably, the ratio of cellulosic sugar hydrolysate to the another sugar hydrolysate obtained from the saccharification of a non-cellulose based sugar source material ranges from 80:20 to 20:80. More preferably, the ratio of cellulosic sugar hydrolysate to the another sugar hydrolysate obtained from the saccharification of a non-cellulose-based sugar source material ranges from 60:40 to 40:60. It is known to those skilled in the art that the ideal ratio will be that which will lead to a larger reduction in carbon intensity metrics while providing cost benefits.

According to a preferred embodiment of the present invention, the use of the cellulosic sugar hydrolysate with the another sugar hydrolysate obtained from the saccharification of a non-cellulose based sugar source material results in at least 5% less carbon intensity score (in gCO₂e/MJ) for the production of the purified value added product than if said purified value added product is produced solely from the another sugar hydrolysate obtained from the saccharification of a non-cellulose based sugar source material. Preferably, the reduction in carbon intensity score is more than 10%. More preferably, the reduction in carbon intensity score is more than 20%.

According to a preferred embodiment of the present invention, the high purity cellulose comprises less than 15% of hemicellulose. Preferably, the high purity cellulose comprises less than 10% of hemicellulose. More preferably, the high purity cellulose comprises less than 5% of hemicellulose. According to a preferred embodiment of the present invention, the high purity cellulose may comprise hemicellulose as during the step of fermenting said combined hydrolysate stream, the fermentation organism(s) may be either able to ferment such into value added products or be able to be engineered to ferment such into value added products. According to a preferred embodiment, one way to ferment C5 sugars from hemicellulose is by engineering an organism that already ferments C6 sugars (from cellulose) to additionally ferment C5 sugars (from hemicellulose) (i.e., engineered yeasts). According to another preferred embodiment, one can employ co-fermentation using 2 organisms: one able to ferment C6 (cellulose) and one able to ferment C5 (hemicellulose).

According to a preferred embodiment of the present invention, the sugar hydrolysate obtained from the saccharification of a non-cellulose based sugar source material is obtained from the saccharification of non-cellulose based sugar source materials including, but not limited to, sugar crops and grains (starches, cereals), such as corn, corn fiber, molasses, sugar beets, sugar cane, sweet sorghum, wheat, cassava, rye, potatoes, sorghum grain, barley, their corresponding waste materials and/or combinations thereof.

Those skilled in the art know that different value-added products can be obtained from the fermentation of sugar extracts or hydrolysates. The different value-added products are obtained when different reaction conditions or fermenting organisms are employed. Examples of value-added products obtained from the fermentation of the hydrolysate obtained in the present invention include, but are not limited to, organic acids (i.e., formic acid, acetic acid), alcohols (i.e., ethanol, isopropanol, isobutanol, n-butanol, propanol), ketones (i.e., acetone), and combinations thereof. According to a preferred embodiment of the present invention, the value-added product is sorbitol. According to a preferred embodiment of the present invention, the value-added product is ethanol. According to another preferred embodiment of the present invention, the value-added product is hydroxymethylfurfural. According to another preferred embodiment of the present invention, the value-added product is selected from the group consisting: levulinic acid; chloromethylfurfural; and 2,5-furandicarboxylic acid.

In some embodiments of the present invention, the process may further include a step to separate and subsequently purify the value-added product from the rest of the fermentation stream.

According to a preferred embodiment of the present invention, the high purity cellulose is a cellulose that has not undergone any distinct bleaching steps, such as a bleaching of a pulp.

According to a preferred embodiment of the present invention, the method of delignification of the lignocellulosic biomass material which yields a high purity cellulose (also referred to as low kappa number cellulose and also referred to as modified Caro's acid delignified cellulose) used in the production a low carbon intensive fermentation stream comprise:
the source of cellulose is a lignocellulosic biomass delignified by exposure to a modified Caro's acid composition having a pH of less than 1, said modified Caro's acid composition selected from the group consisting of: composition A; composition B; composition C; composition D; composition E; composition F; composition G; composition H; composition I; and composition J;
   wherein said composition A comprises:
sulfuric acid;
a compound comprising an amine moiety and a sulfonic acid moiety; and
a peroxide; and wherein sulfuric acid, said compound comprising an amine moiety and a sulfonic acid moiety and said peroxide are present in a molar ratio of no less than 1:1:1;
   wherein said composition B comprises:
sulfuric acid;
a compound comprising an amine moiety;
a compound comprising a sulfonic acid moiety; and
a peroxide; wherein sulfuric acid and said a compound comprising an amine moiety and said compound comprising a sulfonic acid moiety are present in a molar ratio of no less than 1:1:1;
   wherein said composition C comprises:
an alkylsulfonic acid; and
a peroxide; wherein said alkylsulfonic acid and said peroxide are present in a molar ratio of no less than 1:1;
   wherein said composition D comprises:
sulfuric acid;
a heterocyclic compound; and
a peroxide; and wherein sulfuric acid and said a heterocyclic compound; are present in a molar ratio of no less than 1:1;
   wherein said composition E comprises:
sulfuric acid;
a modifying agent comprising a compound containing an amine group; and
a peroxide; and wherein sulfuric acid and said compound containing an amine group; are present in a molar ratio of no less than 1:1;
   wherein said composition F comprises:
sulfuric acid;
a modifying agent comprising an alkanesulfonic acid and
a peroxide; and wherein sulfuric acid and said alkanesulfonic acid are present in a molar ratio of no less than 1:1;
   wherein said composition G comprises:
sulfuric acid;
a substituted aromatic compound; and
a peroxide; and wherein sulfuric acid and said substituted aromatic compound; are present in a molar ratio of no less than 1:1;
   wherein said composition H comprises:
sulfuric acid;
a modifying agent comprising an arylsulfonic acid;
a peroxide; and
optionally, a compound containing an amine group; wherein sulfuric acid and said a arylsulfonic acid; are present in a molar ratio of no less than 1:1;
   wherein said composition I comprises:
sulfuric acid;
a heterocyclic compound;
an alkanesulfonic acid and
a peroxide; and wherein sulfuric acid and said a heterocyclic compound; are present in a molar ratio of no less than 1:1;
   wherein said composition J comprises:
sulfuric acid;
a carbonyl-containing nitrogenous base compound; and
a peroxide; and wherein sulfuric acid and said a carbonyl-containing nitrogenous base compound; are present in a molar ratio of no less than 1:1;
   for a period of time sufficient to remove substantially all of the lignin present on said biomass material. To alleviate the text, the above-described process can be referred to hereinafter as the modified Caro's acid delignification process, as well as the obtained cellulose can be referred to as modified Caro's acid delignified cellulose or “MCA cellulose” to indicate the method of delignification employed to obtain said cellulose.

Preferably, said sulfuric acid, said compound comprising an amine moiety and a sulfonic acid moiety and said peroxide are present in a molar ratio of no more than 15:1:1. Also preferably, said sulfuric acid and said compound comprising an amine moiety and a sulfonic acid moiety are present in a molar ratio of no less than 3:1.

According to a preferred embodiment of the approach to obtain a low hemicellulose content and low lignin cellulose, said delignification lasts from 2 to 20 hours.

According to a preferred embodiment of the approach to obtain low hemicellulose content and low lignin cellulose, said delignification is carried out at temperatures below 50° C. Preferably, the delignification is carried out at temperatures below 40° C.

According to a preferred embodiment of the present invention, the process generates a value-added product such as ethanol from a combination of sugar and starch materials and a high purity cellulose. Said high purity cellulose being defined as having a low Kappa number and low hemicellulose content. Preferably, the combination of a sugar hydrolysate stream from each process significantly decreases the carbon intensity score of the value-added product generated when compared to the process where only the sugar or starch material is employed.

### DETAILED DESCRIPTION OF THE INVENTION

The description that follows, and the embodiments described therein, is provided by way of illustration of an example, or examples, of particular embodiments of the principles of the present invention. These examples are provided for the purposes of explanation, and not limitation, of those principles and of the invention.

According to an embodiment of the present invention, there is provided a process to manufacture a value added product by blending a stream of cellulose-based hydrolysate with a non-cellulose based hydrolysate, wherein said process comprising the steps of:
providing a high purity cellulose comprising of less than 1.5% lignin;
   exposing said high purity cellulose to a saccharification process to produce a cellulosic hydrolysate comprising sugars obtained from the hydrolysis of cellulose and hemicellulose;
   exposing said cellulosic hydrolysate to another sugar hydrolysate obtained from a saccharification of a non-cellulose based sugar source material, thus obtaining a combined hydrolysate stream;
   processing said combined hydrolysate stream to produce said value-added products; and
   optionally, purifying and/or separating said at least one value-added product from the rest of the fermentation stream to yield a purified value-added product.

According to an aspect of the present invention, there is provided a process to manufacture a value added product by blending a stream of cellulose-based hydrolysate with a non-cellulose based hydrolysate, wherein said process comprising the steps of:
providing a lignocellulosic biomass;
exposing said lignocellulosic biomass to a delignification process whereby a high purity cellulose is obtained; said high purity cellulose comprising of less than 1.5% lignin;
exposing said high purity cellulose to a saccharification process to produce a cellulosic hydrolysate comprising sugars obtained from the hydrolysis of cellulose and hemicellulose;
exposing said cellulosic hydrolysate to another sugar hydrolysate obtained from a saccharification of a non-cellulose based sugar source material, thus obtaining a combined hydrolysate stream;
processing said combined hydrolysate stream to produce said value-added products; and
optionally, purifying and/or separating said at least one value-added product from the rest of the fermentation stream to yield a purified value-added product.

According to an aspect of the present invention, there is provided a process to obtain a low carbon intensive combined hydrolysate stream, said process consisting of the steps of:
providing a lignocellulosic biomass;
exposing said lignocellulosic biomass to a modified Caro's acid composition for a period of time necessary to remove more than 98.5% of the lignin present in said lignocellulosic biomass and thus obtaining a high purity cellulose and a liquid stream;
exposing said high purity cellulose to a saccharification process to produce a cellulosic hydrolysate comprising sugars resulting from the hydrolysis of cellulose and hemicellulose;
exposing said cellulosic hydrolysate to another sugar hydrolysate obtained from the saccharification of a non-cellulose-based sugar source, thus obtaining a combined hydrolysate stream;
processing said combined hydrolysate stream to produce said value-added product; and
optionally, purifying said at least one value-added product to yield a purified value-added product.

According to a preferred embodiment of the present invention, said lignocellulosic biomass may be mechanically treated to reduce particle size prior to contacting it to a modified Caro's acid.

In some embodiments of the present invention, the process of exposing said lignocellulosic biomass to a modified Caro's acid composition can be carried out for a varying duration of time depending on the particle size of the biomass and the type of biomass being fed into the process. In some cases, the process can last from 2 to 20 hours depending on that characteristic. The process is preferably run at temperatures below 50° C., more preferably at temperatures below 40° C.

According to a preferred embodiment of the present invention, the high purity cellulose and liquid streams are separated using any type of solid-liquid separation including centrifugation, filtration and/or combinations thereof.

According to a preferred embodiment of the present invention, the cellulosic sugar hydrolysate is combined with the another sugar hydrolysate obtained from the saccharification of a non-cellulose based sugar source material in a weight ratio ranging from 99:1 to 1:99. Preferably, the ratio of cellulosic sugar hydrolysate to the another sugar hydrolysate obtained from the saccharification of a non-cellulose based sugar source material ranges from 80:20 to 20:80. More preferably, the ratio of cellulosic sugar hydrolysate to the another sugar hydrolysate obtained from the saccharification of a non-cellulose-based sugar source material ranges from 60:40 to 40:60. It is known to those skilled in the art that the ideal ratio will be that which will lead to a larger reduction in carbon intensity metrics while providing cost benefits.

According to a preferred embodiment of the present invention, the use of the cellulosic sugar hydrolysate with the another sugar hydrolysate obtained from the saccharification of a non-cellulose based sugar source material results in at least 5% less carbon intensity score (in gCO₂e/MJ) for the production of the purified value added product than if said purified value added product is produced solely from the another sugar hydrolysate obtained from the saccharification of a non-cellulose based sugar source material. Preferably, the reduction in carbon intensity score is more than 10%. More preferably, the reduction in carbon intensity score is more than 20%.

In some embodiments of the present invention, the process may further include a step to recover the liquid stream and upgrade it to value added products including fuels, industrial chemicals and/or energy.

According to another embodiment of the present invention, there is also disclosed a process to obtain a low carbon intensive fermentation stream from cellulose source, said process consisting of the following steps:
providing a reaction vessel;
providing a cellulose source delignified using a modified Caro's acid in an aqueous medium, wherein said cellulose source has a Kappa number of less than 10, more preferably less than 5 and even more preferably, less than 2;
exposing said cellulose source to an enzyme blend that is capable of converting said cellulose source into glucose and other sugars, thus obtaining a cellulosic saccharified solution;
exposing said cellulosic saccharified solution to a sugar hydrolysate obtained from the saccharification of a non-cellulose based sugar source material, thus obtaining a combined hydrolysate stream; and
exposing said saccharified solution to an organism, which converts said saccharified solution to value-added products.

It is believed that a high purity cellulose allows the generation of a substantially pure sugar hydrolysate stream mainly comprised of glucose which can further be combined with a sugar hydrolysate stream coming from a non-cellulose based sugar source material.

It is widely accepted that a Kappa number is a reliable indication of lignin content in a pulp or cellulosic material. The higher the Kappa number, the higher the lignin content is. The Kappa number is a measure of the degree of fibrous pulp digestion and can be applied to determine lignin content. Its value can vary from 0 to over 100, where 0 indicates a practically lignin-free pulp (such as that found in bleached pulp) and where a Kappa number of 60 is usually attained with a standard unbleached pulp. When the Kappa number is 60, this is a rough indication that the lignin content is about 9%, when the Kappa number is about 20, this would indicate a lignin content of approximately 2.8-3.0%. When the Kappa number is about 27, the lignin content is approximately 4.0%.

According to a preferred embodiment of the present invention, the process of exposing said high purity cellulose to a saccharification process to produce a cellulosic hydrolysate comprises at least one of the following methods: the use of an enzyme blend, the use of an organism or combination of organisms, and the use of a chemical blend.

According to a preferred embodiment of the present invention, the pH of the high purity cellulose is adjusted prior to being exposed to a saccharification process. Preferably, the pH is adjusted to any value within the range between 3 to 7. Preferably, the pH is adjusted with any chemical known to those skilled in the art that can neutralize the modified Caro's acid. Preferably, the pH is adjusted with a hydroxide salt, such as ammonium hydroxide or sodium hydroxide.

According to a preferred embodiment of the present invention, said enzyme blend comprises of cellulases and hemicellulases. Preferably, said enzyme blend comprises of at least one exo-glucanase, at least one endo-glucanase and at least one β-glucosidase. More preferably, said enzyme blend also comprises at least one endo-xylanase and at least one β-xylosidase.

According to a preferred embodiment of the present invention, said organism or combination of organisms include prokaryotic and eukaryotic organisms comprising cellulases and hemicellulases. Preferably, said organism is a prokaryotic organism capable of breaking down cellulose and hemicellulose. Said organism can be selected from the group comprising, but not limited to, organisms to the genus Zymomonas, Pseudomonas, Cellulomonas, Trichoderma, Cytophaga, and Aspergillus.

According to another embodiment of the present invention, said chemical blend comprises of an acid or a base or combinations of multiple acids or bases. Preferably, said chemical blend comprises a mineralic acid. Preferably, said chemical blend comprises a dilute hydrochloric acid, orto-phosphoric acid, sulfuric acid, nitric acid, or combinations thereof.

According to a preferred embodiment of the present invention, said aqueous medium has a pH of about 4.0 to 6.0.

According to a preferred embodiment of the present invention, the process of exposing said cellulose source to said enzyme blend occurs at a temperature of less than 70° C. Preferably said process occurs at a temperature between 40 to 60° C. According to a preferred embodiment of the present invention, the process of exposing said cellulose source to said enzyme blend occurs for a period of time ranging from 1 to 168 hours, preferably between 24 and 144 hours, and more preferably between 48 and 120 hours.

The term “saccharified solution” refers to a composition comprising of mostly simple sugars such as oligo, di-, and monosaccharides (i.e., glucose, xylose, etc.). In some embodiments, the term “saccharified solution” might also be referred to compositions where some complex sugars (i.e., polysaccharides including undegraded cellulose and hemicellulose) are present.

In some embodiments of the present invention, different organisms are used in the production of different value-added products from the combined sugar hydrolysate. Said organism can be selected from the group comprising of eukaryotic or prokaryotic organisms, including but not limited to bacteria, archaea, fungi (yeasts and molds), algae, protozoa and/or combinations thereof. As a non-limiting example, Saccharomyces cerevisiae or Zymomonas mobilis will be a preferred organism should the production of ethanol is sought after.

### Process to Obtain a Low Kappa Number Delignified Cellulose

According to a preferred embodiment of the present invention, the method of delignification of biomass material which yields a modified Caro's acid delignified cellulose used in the cellulose to cellobiose (and ultimately, glucose) conversion experiments comprise:
providing a biomass material comprising cellulose fibers and lignin;
exposing said biomass material requiring delignification to a modified Caro's acid composition having a pH of less than 1, said modified Caro's acid composition selected from the group consisting of: composition A; composition B; composition C; composition D; composition E; composition F; composition G; composition H; composition I; and composition J;
   wherein said composition A comprises:
sulfuric acid;
a compound comprising an amine moiety and a sulfonic acid moiety; and
a peroxide; and wherein sulfuric acid, said compound comprising an amine moiety and a sulfonic acid moiety and said peroxide are present in a molar ratio of no less than 1:1:1;
   wherein said composition B comprises:
sulfuric acid;
a compound comprising an amine moiety;
a compound comprising a sulfonic acid moiety; and
a peroxide; wherein sulfuric acid and said a compound comprising an amine moiety and said compound comprising a sulfonic acid moiety are present in a molar ratio of no less than 1:1:1;
   wherein said composition C comprises:
an alkylsulfonic acid; and
a peroxide; wherein said alkylsulfonic acid and said peroxide are present in a molar ratio of no less than 1:1;
   wherein said composition D comprises:
sulfuric acid;
a heterocyclic compound; and
a peroxide; and wherein sulfuric id and said a heterocyclic compound; are present in a molar ratio of no less than 1:1;
   wherein said composition E comprises:
sulfuric acid;
a modifying agent comprising a compound containing an amine group; and
a peroxide; and wherein sulfuric acid and said compound containing an amine group; are present in a molar ratio of no less than 1:1;
   wherein said composition F comprises:
sulfuric acid;
a modifying agent comprising an alkanesulfonic acid and
a peroxide; and wherein sulfuric acid and said alkanesulfonic acid are present in a molar ratio of no less than 1:1;
   wherein said composition G comprises:
sulfuric acid;
a substituted aromatic compound; and
a peroxide; and wherein sulfuric acid and said substituted aromatic compound; are present in a molar ratio of no less than 1:1;
   wherein said composition H comprises:
sulfuric acid;
a modifying agent comprising an arylsulfonic acid;
a peroxide; and
optionally, a compound containing an amine group; wherein sulfuric acid and said a arylsulfonic acid; are present in a molar ratio of no less than 1:1;
   wherein said composition I comprises:
sulfuric acid;
a heterocyclic compound;
an alkanesulfonic acid and
a peroxide; and wherein sulfuric acid and said a heterocyclic compound; are present in a molar ratio of no less than 1:1;
   wherein said composition J comprises:
sulfuric acid;
a carbonyl-containing nitrogenous base compound; and
a peroxide; and wherein sulfuric acid and said a carbonyl-containing nitrogenous base compound; are present in a molar ratio of no less than 1:1;

for a period of time sufficient to remove substantially all of the lignin present on said biomass material. The process can be carried out for a varying duration of time depending on the particle size of the biomass being fed into the process. The process can last from 2 to 20 hours depending on that characteristic. Moreover, the temperature of the resulting mixture also has an impact on the duration of the process as the reaction is highly exothermic, precautions are taken to prevent a runaway degradation of the cellulose. This would result in a carbon black resulting product with no value. The process is preferably run at temperatures below 50° C., more preferably at temperatures below 40° C. The process of delignification is preferably performed with a cooling means adapted to control the heat generated by the chemical reaction of delignification and maintain the temperature to avoid an undesirable ‘runaway’ reaction.

Preferably, said sulfuric acid, said compound comprising an amine moiety and a sulfonic acid moiety and said peroxide are present in a molar ratio of no more than 15:1:1. Preferably, for a modified Caro's acid comprising sulfuric acid, peroxide and taurine (as the modifier component), the molar composition is as follows: H₂O:H₂O₂:H₂SO₄:Taurine in a molar ratio of 56:10: 10:1. Preferably, for a modified Caro's acid comprising TEOA/MSA, the molar composition is as follows: H₂O:H₂O₂:H₂SO₄:TEOA:MSA in a molar ratio of 56:10:10:1:1.

According to a preferred embodiment of the approach to obtain low lignin cellulose, said sulfuric acid and said compound comprising an amine moiety and a sulfonic acid moiety are present in a molar ratio of no less than 3:1.

Preferably, said compound comprising an amine moiety and a sulfonic acid moiety is selected from the group consisting of: taurine; taurine derivatives; and taurine-related compounds.

According to a preferred embodiment of the approach to obtain low lignin cellulose, said taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates; as well as aminoalkylsulfonic acids where the alkyl is selected from the group consisting of C₁-C₅ linear alkyl and C₁-C₅ branched alkyl.

Preferably, said linear alkylaminosulfonic acid is selected form the group consisting of: methyl; ethyl (taurine); propyl; and butyl.

Preferably, branched aminoalkylsulfonic acid is selected from the group consisting of: isopropyl; isobutyl; and isopentyl.

According to a preferred embodiment of the approach to obtain low lignin cellulose, said compound comprising an amine moiety and a sulfonic acid moiety is taurine.

According to a preferred embodiment of the approach to obtain low lignin cellulose, said sulfuric acid and compound comprising an amine moiety and a sulfonic acid moiety are present in a molar ratio of no less than 3:1.

According to a preferred embodiment of the approach to obtain low lignin cellulose, said compound comprising an amine moiety is an alkanolamine is selected from the group consisting of: monoethanolamine; diethanolamine; triethanolamine; and combinations thereof.

According to a preferred embodiment of the approach to obtain low lignin cellulose, said compound comprising a sulfonic acid moiety is selected from the group consisting of: alkylsulfonic acids; arylsulfonic acids; and combinations thereof.

Preferably, said alkylsulfonic acid is selected from the group consisting of: alkylsulfonic acids where the alkyl groups range from C₁-C₆ and are linear or branched; and combinations thereof. More preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof.

Preferably, said arylsulfonic acid is selected from the group consisting of: toluenesulfonic acid; benzesulfonic acid; and combinations thereof.

According to a preferred embodiment of the approach to obtain low lignin cellulose, said alkylsulfonic acid; and said peroxide are present in a molar ratio of no less than 1:1.

Preferably, said compound comprising a sulfonic acid moiety is methanesulfonic acid.

According to a preferred embodiment of the approach to obtain low lignin cellulose (i.e. MCA cellulose), said Composition C may further comprise a compound comprising an amine moiety. Preferably, the compound comprising an amine moiety has a molecular weight below 300 g/mol. Preferably also, the compound comprising an amine moiety is a primary amine. More preferably, the compound comprising an amine moiety is an alkanolamine. Preferably, the compound comprising an amine moiety is a tertiary amine. According to a preferred embodiment of the approach to obtain low lignin cellulose, the alkanolamine is selected from the group consisting of: monoethanolamine; diethanolamine; triethanolamine; and combinations thereof. Preferably, the alkanolamine is triethanolamine.

According to a preferred embodiment of the approach to obtain low lignin cellulose, said in Composition C, said sulfuric acid and said a compound comprising an amine moiety and said compound comprising a sulfonic acid moiety are present in a molar ratio of no less than 1:1:1.

Preferably, in Composition C, said sulfuric acid, said compound comprising an amine moiety and said compound comprising a sulfonic acid moiety are present in a molar ratio ranging from 28:1:1 to 2:1:1.

Preferably, in Composition C, said compound comprising an amine moiety is triethanolamine and said compound comprising a sulfonic acid moiety is methanesulfonic acid.

According to preferred embodiment of the present invention, the modified Caro's acid (as disclosed in Canadian patent application 3,128,678) comprises: sulfuric acid; a heterocyclic compound and a peroxide; and wherein sulfuric acid and said a heterocyclic compound; are present in a molar ratio of no less than 1:1. Preferably, the sulfuric acid and said heterocyclic compound are present in a molar ratio ranging from 28:1 to 2:1 More preferably, the sulfuric acid and heterocyclic compound are present in a molar ratio ranging from 24:1 to 3:1. Preferably, the sulfuric acid and heterocyclic compound are present in a molar ratio ranging from 20:1 to 4:1. More preferably, the sulfuric acid and heterocyclic compound are present in a molar ratio ranging from 16:1 to 5:1. Preferably, the sulfuric acid and heterocyclic compound are present in a molar ratio ranging from 12:1 to 6:1. Also preferably, said heterocyclic compound has a molecular weight below 300 g/mol. Also preferably, said heterocyclic compound has a molecular weight below 150 g/mol. More preferably, said heterocyclic compound is a secondary amine. According to a preferred embodiment of the present invention, said heterocyclic compound is selected from the group consisting of: imidazole; triazole; and N-methylimidazole.

According to preferred embodiment of the present invention, the modified Caro's acid (as disclosed in Canadian patent application 3,128,677) comprises: sulfuric acid; a modifying agent comprising a compound containing an amine group and a peroxide; and wherein sulfuric acid and said compound containing an amine group; are present in a molar ratio of no less than 1:1. Preferably, the sulfuric acid and said compound containing an amine group are present in a molar ratio ranging from 28:1 to 2:1. More preferably, the sulfuric acid and compound containing an amine group are present in a molar ratio ranging from 24:1 to 3:1. Preferably, the sulfuric acid and compound containing an amine group are present in a molar ratio ranging from 20:1 to 4:1. More preferably, the sulfuric acid and compound containing an amine group are present in a molar ratio ranging from 16:1 to 5:1. Preferably, the sulfuric acid and compound containing an amine group are present in a molar ratio ranging from 12:1 to 6:1. According to a preferred embodiment of the present invention, the modifying agent is selected in the group consisting of: TEOA; MEOA; pyrrolidine; DEOA; ethylenediamine; diethylamine; triethylamine; morpholine; MEA-triazine; and combinations thereof. According to a more preferred embodiment of the present invention, the modifying agent is TEOA; MEOA; pyrrolidine; DEOA; ethylenediamine; triethylamine.

According to preferred embodiment of the present invention, the modified Caro's acid (as disclosed in Canadian patent application 3,128,676) comprises: sulfuric acid; a modifying agent comprising an alkanesulfonic acid and a peroxide; and wherein sulfuric acid and said alkanesulfonic acid are present in a molar ratio of no less than 1:1. Preferably, said alkanesulfonic acid is selected from the group consisting of: alkanesulfonic acids where the alkyl groups range from C₁-C₆ and are linear or branched; and combinations thereof. Preferably, said alkanesulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. More preferably, said alkanesulfonic acid is methanesulfonic acid. Also preferably, said alkanesulfonic acid has a molecular weight below 300 g/mol. Also preferably, said alkanesulfonic acid has a molecular weight below 150 g/mol. Preferably, the sulfuric acid and said alkanesulfonic acid and are present in a molar ratio ranging from 28:1 to 2:1. More preferably, the sulfuric acid and alkanesulfonic acid are present in a molar ratio ranging from 24:1 to 3:1. Preferably, the sulfuric acid and alkanesulfonic acid are present in a molar ratio ranging from 20:1 to 4:1. More preferably, the sulfuric acid and alkanesulfonic acid are present in a molar ratio ranging from 16:1 to 5:1. According to a preferred embodiment of the present invention, the sulfuric acid and alkanesulfonic acid are present in a molar ratio ranging from 12:1 to 6:1.

According to preferred embodiment of the present invention, the modified Caro's acid (as disclosed in Canadian patent application 3,128,675) comprises: sulfuric acid; a substituted aromatic compound and a peroxide; and wherein sulfuric acid and said substituted aromatic compound; are present in a molar ratio of no less than 1:1. Preferably, the substituted aromatic compound comprises at least two substituents. More preferably, at least one substituent is an amine group and at least one of the other substituent is a sulfonic acid moiety. According to a preferred embodiment, the substituted aromatic compound comprises three or more substituent. According to a preferred embodiment of the present invention, the substituted aromatic compound comprises at least a sulfonic acid moiety. According to another preferred embodiment of the present invention, the substituted aromatic compound comprises an aromatic compound having a sulfonamide substituent, where the compound can be selected from the group consisting of: benzenesulfonamides; toluenesulfonamides; substituted benzenesulfonamides; and substituted toluenesulfonamides. Preferably, the sulfuric acid and said substituted aromatic compound and are present in a molar ratio ranging from 28:1 to 2:1. More preferably, the sulfuric acid and substituted aromatic compound are present in a molar ratio ranging from 24:1 to 3:1. Preferably, the sulfuric acid and substituted aromatic compound are present in a molar ratio ranging from 20:1 to 4:1. More preferably, the sulfuric acid and substituted aromatic compound are present in a molar ratio ranging from 16:1 to 5:1. Preferably, the sulfuric acid and substituted aromatic compound are present in a molar ratio ranging from 12:1 to 6:1.

According to preferred embodiment of the present invention, the modified Caro's acid (as disclosed in Canadian patent application 3,128,674) comprises: sulfuric acid; a modifying agent comprising an arylsulfonic acid; a peroxide; and optionally, a compound containing an amine group; wherein sulfuric acid and said a arylsulfonic acid; are present in a molar ratio of no less than 1:1. Preferably, the compound containing an amine group is selected from the group consisting of: imidazole; N-methylimidazole; triazole; monoethanolamine (MEOA); diethanolamine (DEOA); triethanolamine (TEOA); pyrrolidine and combinations thereof. According to a preferred embodiment of the present invention, sulfuric acid and the peroxide are present in a molar ratio of approximately 1:1. Preferably, the sulfuric acid and said arylsulfonic acid and are present in a molar ratio ranging from 28:1 to 2:1. More preferably, the sulfuric acid and arylsulfonic acid are present in a molar ratio ranging from 24:1 to 3:1. Preferably, the sulfuric acid and arylsulfonic acid are present in a molar ratio ranging from 20:1 to 4:1. More preferably, the sulfuric acid and arylsulfonic acid are present in a molar ratio ranging from 16:1 to 5:1. According to a preferred embodiment of the present invention, the sulfuric acid and arylsulfonic acid are present in a molar ratio ranging from 12:1 to 6:1. Also preferably, said arylsulfonic acid has a molecular weight below 300 g/mol. Also preferably, said arylsulfonic acid has a molecular weight below 150 g/mol. Even more preferably, said arylsulfonic acid is selected from the group consisting of: orthanilic acid; metanilic acid; sulfanilic acid; toluenesulfonic acid; benzenesulfonic acid; and combinations thereof.

According to preferred embodiment of the present invention, the modified Caro's acid (as disclosed in Canadian patent application 3,128,673) comprises: sulfuric acid; a heterocyclic compound; an alkanesulfonic acid and a peroxide; and wherein sulfuric acid and said a heterocyclic compound; are present in a molar ratio of no less than 1:1. Preferably, said aqueous acidic composition comprising: sulfuric acid; a heterocyclic compound; an arylsulfonic acid; and wherein sulfuric acid and said a heterocyclic compound; are present in a molar ratio of no less than 1:1. Preferably, the arylsulfonic acid is toluenesulfonic acid.

Preferably, the sulfuric acid, the heterocyclic compound and the alkanesulfonic acid are present in a molar ratio ranging from 28:1:1 to 2:1:1. More preferably, the sulfuric acid the heterocyclic compound and the alkanesulfonic acid are present in a molar ratio ranging from 24:1:1 to 3:1:1. Preferably, the sulfuric acid, the heterocyclic compound and the alkanesulfonic acid are present in a molar ratio ranging from 20:1:1 to 4:1:1. More preferably, the sulfuric acid, the heterocyclic compound and the alkanesulfonic acid are present in a molar ratio ranging from 16:1:1 to 5:1:1. According to a preferred embodiment of the present invention, the sulfuric acid and heterocyclic compound are present in a molar ratio ranging from 12:1:1 to 6:1:1. Also preferably, said heterocyclic compound has a molecular weight below 300 g/mol. Also preferably, said heterocyclic compound has a molecular weight below 150 g/mol. Even more preferably, said heterocyclic compound is selected from the group consisting of: imidazole; triazole; n-methylimidazole; and combinations thereof. Preferably, the alkanesulfonic acid is selected from the group consisting of: alkylsulfonic acids where the alkyl groups range from C₁-C₆ and are linear or branched; and combinations thereof. Preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. More preferably, said alkylsulfonic acid is methanesulfonic acid.

According to preferred embodiment of the present invention, the modified Caro's acid (as disclosed in Canadian patent application 3,128,672) comprises: sulfuric acid; a carbonyl-containing nitrogenous base compound and a peroxide; and wherein sulfuric acid and said a carbonyl-containing nitrogenous base compound; are present in a molar ratio of no less than 1:1. According to a preferred embodiment of the present invention, the carbonyl-containing nitrogenous base compound is selected from the group consisting of: caffeine; lysine; creatine; glutamine; creatinine; 4-aminobenzoic acid; glycine; NMP (N-methyl-2-pyrrolidinone); histidine; DMA (N,N-dimethylacetamide); arginine; 2.3-pyridinedicarboxylic acid; hydantoin; and combinations thereof. Preferably, the sulfuric acid and said carbonyl-containing nitrogenous base compound and are present in a molar ratio ranging from 28:1 to 2:1. More preferably, the sulfuric acid and carbonyl-containing nitrogenous base compound are present in a molar ratio ranging from 24:1 to 3:1. Preferably, the sulfuric acid and carbonyl-containing nitrogenous base compound are present in a molar ratio ranging from 20:1 to 4:1. More preferably, the sulfuric acid and carbonyl-containing nitrogenous base compound are present in a molar ratio ranging from 16:1 to 5:1. According to a preferred embodiment of the present invention, the sulfuric acid and carbonyl-containing nitrogenous base compound are present in a molar ratio ranging from 12:1 to 6:1.

### Experiment #1—Lifecycle Assessment (LCA)

A life cycle assessment (LCA) was conducted on a process according to a preferred embodiment of the present invention using guidelines from ISO 14040/4044/4064 to determine the carbon intensity score (CI score) of the cellulose. The process comprises a step of generating a cellulose source, said cellulose source being a lignocellulosic material having undergone delignification using a modified Caro's acid in an aqueous medium, wherein said cellulose source has a Kappa number of less than 10, more preferably less than 5 and even more preferably, less than 2. Preferably, the hemicellulose content of said cellulose source was less than 15% wt. of the total weight of the cellulose source.

The carbon intensity (CI) of generic 1G ethanol varies between 30-70 g CO₂e/MJ, typically in North America values are on the higher range due to the use of corn and wheat biomass. Biomass source plays a major factor as well as the type of utilities being used in the process. Other factors such as efficient farming practices, utilization of residues, transportation networks, and process efficiency also play a role in the CI. A cradle-to-gate approach was taken in the LCA analysis of the production of the high purity cellulose as described in the present invention, which accounted for raw material sources, process mass and energy balance. Based on today's green chemical supply chain, this yielded a CI score of approximately 40-50 g CO₂eq/MJ for the cellulose, dependent on biomass feedstock selection, canola straw was used in the study. Given that the process to convert cellulose to glucose happens at near ambient operating conditions with minimal use of raw inputs. The enzymatic saccharification process approximately emits 2 g CO₂eq/MJ yielding a final CI score of 42-52 g CO₂eq/MJ for glucose derived from a lignocellulosic biomass delignified using a modified Caro's acid as discussed hereinabove. This value will greatly decrease in the near future due to the global energy transition, potentially yielding as low as 5-10 g CO₂e/MJ once commercial infrastructure and supply chain is established.

### Experiment #2—Combined Fermentation of Hydrolysates Derived from 1G and 2G Saccharifications Using Corn Starch and Cellulose

Corn starch and a high purity cellulose obtained from the delignification using a modified Caro's acid were employed for this experiment. After saccharification, both hydrolysates were filtered and tested for sugars content using an HPLC coupled with a refractive index detector. Various ratios of the 1G and 2G hydrolysates were combined based on their sugar content in order to obtain a final sugar concentration of 15% (w/w). Saccharomyces cerevisiae was added to the combined solutions at a loading of 0.2% (w/w) and fermented for 93 hours at 30° C.

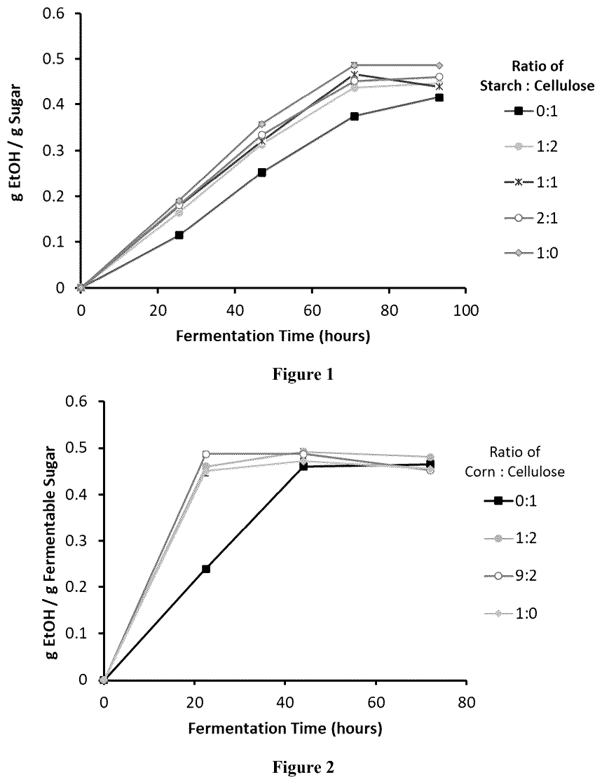

As seen in FIG. 1, ethanol yields (g EtOH/g sugar) were comparable across all 1G+2G ratios of saccharified starch and cellulose combinations, with only minor variations in overall performance. Fermentation of the purely starch-based hydrolysate (1:0) resulted in a yield of 0.49 g EtOH/g sugar after 4 days of fermentation; while the fermentation of the pure cellulosic hydrolysate (0:1) reached 0.42 g EtOH/g sugar at the same time point. However, this still represents 85% of the yield observed for the starch hydrolysate, demonstrating that with the appropriate time, the fermentation of saccharified cellulose is comparable to that of pure starch for ethanol production. The mixed starch-cellulose hydrolysates (1:1, 1:2, and 2:1 ratios) exhibited ethanol yields that closely followed those of pure starch-based sugars (FIG. 1). The relatively small differences across varying compositions of sugar solutions indicate that co-fermentation of starch and cellulose can be an effective strategy for bioethanol production without significant loss in efficiency.

The results from this Experiment show how starch-based hydrolysates are able to be combined with cellulosic-based hydrolysates to provide comparable yields when the cellulose used for the saccharification has been obtained through a delignification reaction using a modified Caro's acid and is of high quality as described herein. This strategy allows asset owners to increase their ethanol production capacity while lowering their carbon footprint and reducing its reliance on starch-based ethanol. Additionally, the carbon footprint may further reduced due to the characteristics of a delignification described herein wherein ambient temperatures and pressures are utilized throughout the process, reducing the energy consumption of the biomass fractionation step.

### Experiment #3—Combined Fermentation of Hydrolysates Derived from 1G and 2G Saccharifications Using Corn and Cellulose

Corn kernels underwent a dry milling process, followed by cooking/gelatinization and saccharification yielding a 1G saccharified solution. The high purity cellulose produced from a delignification process described herein was also saccharified to obtain a cellulosic-based hydrolysate yielding a 2G saccharified solution.

Both the 1G and 2G saccharified solutions were filtered and tested for sugar content as described above. Various ratios of the sugar hydrolysates were then combined in order to obtain a final sugar concentration of 10% (w/w). Saccharomyces cerevisiae was added to the combined solutions at a loading of 0.2% (w/w) of the total volume and fermented for 72 hours at 30° C.

FIG. 2 demonstrates the ethanol yield as expressed by grams of EtOH per gram of initial simple fermentable sugar. After 44 hours of fermentation, both the purely corn-based hydrolysate and purely cellulose-based hydrolysate solutions reached a comparable ethanol yield of 0.47 grams of ethanol per gram of sugar. The cellulose-based hydrolysate fermentation initially showed a slower ethanol production rate but ultimately reached the same yield as the fermentation of the corn-based hydrolysate. Likewise, all the mixed corn-cellulose hydrolysates exhibited similar yields, indicating that incorporating cellulose into the fermentation process does not negatively impact ethanol production and may, in some cases, enhance efficiency.

The embodiments described herein are to be understood to be exemplary and numerous modification and variations of the present invention are possible in light of the above teachings. It is therefore to be understood that within the scope of the claims appended hereto, the invention may be practiced otherwise than as specifically disclosed herein.

1. A process to manufacture a value added product by blending a stream of cellulose-based hydrolysate with a non-cellulose based hydrolysate, wherein said process comprising the steps of:
providing a high purity cellulose comprising of less than 1.5 wt. % lignin;
exposing said high purity cellulose to a saccharification process to produce a cellulosic hydrolysate comprising sugars obtained from the hydrolysis of cellulose and hemicellulose;
exposing said cellulosic hydrolysate to another sugar hydrolysate obtained from a saccharification of a non-cellulose based sugar source material, thus obtaining a combined hydrolysate stream;
processing said combined hydrolysate stream to produce said value-added product; and
optionally, purifying and/or separating said at least one value-added product from the rest of the fermentation stream to yield a purified value-added product.

2. A process to manufacture a value added product by blending a stream of cellulose-based hydrolysate with a non-cellulose based hydrolysate, wherein said process comprising the steps of:
providing a lignocellulosic biomass;
exposing said lignocellulosic biomass to a delignification process whereby a high purity cellulose is obtained; said high purity cellulose comprising of less than 1.5 wt. % lignin;
exposing said high purity cellulose to a saccharification process to produce a cellulosic hydrolysate comprising sugars obtained from the hydrolysis of cellulose and hemicellulose;
exposing said cellulosic hydrolysate to another sugar hydrolysate obtained from a saccharification of a non-cellulose based sugar source material, thus obtaining a combined hydrolysate stream;
processing said combined hydrolysate stream to produce said value-added product; and
optionally, purifying and/or separating said at least one value-added product from the rest of the fermentation stream to yield a purified value-added product.

3. The process according to claim 1 wherein said high purity cellulose source comprises less than 15% of hemicellulose.

4. A process to obtain a low carbon intensive combined hydrolysate stream, said process comprising the steps of:
providing a lignocellulosic biomass;
exposing said lignocellulosic biomass to a modified Caro's acid composition for a period of time necessary to remove more than 98.5% of the lignin present in said lignocellulosic biomass and thus obtaining a high purity cellulose and a liquid stream;
exposing said high purity cellulose to a saccharification process to produce a cellulosic hydrolysate comprising sugars resulting from the hydrolysis of cellulose and hemicellulose;
exposing said cellulosic hydrolysate to another sugar hydrolysate obtained from the saccharification of a non-cellulose-based sugar source, thus obtaining a combined hydrolysate stream;
processing said combined hydrolysate stream to produce at least one value-added product; and
optionally, purifying said at least one value-added product to yield a purified value-added product.

5. The process according to claim 4 wherein the high purity cellulose comprises less than 15% of hemicellulose.

6. The process according to claim 4 wherein the high purity cellulose has a Kappa number of less than 10.

7. The process according to claim 4 wherein the cellulosic hydrolysate is combined with the another sugar hydrolysate obtained from the saccharification of a non-cellulose based sugar source material in a weight ratio ranging from 99:1 to 1:99.

8. The process according to claim 4 wherein the cellulosic hydrolysate is combined with the another sugar hydrolysate obtained from the saccharification of a non-cellulose based sugar source material in a weight ratio ranging from 80:20 to 20:80.

9. The process according to claim 4 wherein the cellulosic hydrolysate is combined with the another sugar hydrolysate obtained from the saccharification of a non-cellulose based sugar source material in a weight ratio ranging from 60:40 to 40:60.

10. The process according to claim 4 wherein said value-added product is ethanol.

11. The process according to claim 4 wherein said modified Caro's acid composition has a pH of less than 1 and is selected from the group consisting of: composition A; composition B; composition C; composition D; composition E; composition F; composition G; composition H; composition I; and composition J;
wherein said composition A comprises:
sulfuric acid;
a compound comprising an amine moiety and a sulfonic acid moiety; and
a peroxide; and wherein sulfuric acid, said compound comprising an amine moiety and a sulfonic acid moiety and said peroxide are present in a molar ratio of no less than 1:1:1;
wherein said composition B comprises:
sulfuric acid;
a compound comprising an amine moiety;
a compound comprising a sulfonic acid moiety; and
a peroxide; wherein sulfuric acid and said a compound comprising an amine moiety and said compound comprising a sulfonic acid moiety are present in a molar ratio of no less than 1:1:1;
wherein said composition C comprises:
an alkylsulfonic acid; and
a peroxide; wherein said alkylsulfonic acid and said peroxide are present in a molar ratio of no less than 1:1;
wherein said composition D comprises:
sulfuric acid;
a heterocyclic compound; and
a peroxide; and wherein sulfuric acid and said a heterocyclic compound; are present in a molar ratio of no less than 1:1;
wherein said composition E comprises:
sulfuric acid;
a modifying agent comprising a compound containing an amine group; and
a peroxide; and wherein sulfuric acid and said compound containing an amine group; are present in a molar ratio of no less than 1:1;
wherein said composition F comprises:
sulfuric acid;
a modifying agent comprising an alkanesulfonic acid and
a peroxide; and wherein sulfuric acid and said alkanesulfonic acid are present in a molar ratio of no less than 1:1;
wherein said composition G comprises:
sulfuric acid;
a substituted aromatic compound; and
a peroxide; and wherein sulfuric acid and said substituted aromatic compound;
are present in a molar ratio of no less than 1:1;
wherein said composition H comprises:
sulfuric acid;
a modifying agent comprising an arylsulfonic acid;
a peroxide; and
optionally, a compound containing an amine group; wherein sulfuric acid and said a arylsulfonic acid; are present in a molar ratio of no less than 1:1;
wherein said composition I comprises:
sulfuric acid;
a heterocyclic compound;
an alkanesulfonic acid and
a peroxide; and wherein sulfuric acid and said a heterocyclic compound; are present in a molar ratio of no less than 1:1;
wherein said composition J comprises:
sulfuric acid;
a carbonyl-containing nitrogenous base compound; and
a peroxide; and wherein sulfuric acid and said a carbonyl-containing nitrogenous base compound; are present in a molar ratio of no less than 1:1.

12. The process according to claim 4 wherein said saccharification process comprises at least one of the following methods: the use of an enzyme blend, an organism or combination of organisms, and a chemical blend.

13. The process according to claim 12 wherein said enzyme blend comprises of cellulases and hemicellulases.

14. The process according to claim 12 wherein said enzyme blend comprises at least one exo-glucanase, at least one endo-glucanase and at least one β-glucosidase.

15. The process according to claim 4 wherein said saccharification process occurs for a period of 1 to 168 hours.

16. The process according to claim 4 wherein said saccharification process is carried out at a temperature up to 120° C.

17. The process according to claim 4 wherein at least 5% of said cellulosic hydrolysate is obtained through the use of an enzyme blend.
